(19) United States
(12) Patent Application Publication
(10) Pub. No.: US 2004/0215220 A1
(43) Pub. Date: Oct. 28, 2004

(54) Anastomotic stent, apparatus and methods of use thereof

(76) Inventors: Mark J. Dolan, Santa Rosa, CA (US); Jeffrey W. Allen, Santa Rosa, CA (US); Sean T. Saint, Santa Rosa, CA (US)

Correspondence Address: MEDTRONIC AVE, 3576 UNOCAL PLACE, SANTA ROSA, CA, 95403, US

(21) Appl. No.: 10/422,635

(22) Filed: Apr. 24, 2003

(51) Int. Cl. A61B 17/08

(52) U.S. Cl. 606/153; 623/1.15; 623/1.11

(57) ABSTRACT

The present invention provides an anastomotic apparatus for a bypass, including an anastomotic stent with a side port, a bridge conduit including a first end and a second end, and a second anastomotic stent including a side port. The first end of the bridge conduit is in communication with the side port of the first anastomotic stent, and the second end of the bridge conduit is in communication with the side port of the second anastomotic stent. The first anastomotic stent is positionable in a first vessel and the second anastomotic stent is positionable in a second vessel, and the bridge conduit provides a pathway for fluid flow between the first vessel and the second vessel.

### FIELD OF THE INVENTION

This invention relates generally to the field of biomedical stents. More specifically, the invention relates to anastomotic stents with side ports for coronary and peripheral bypasses, and methods of use.

### BACKGROUND OF THE INVENTION

The human body has numerous vessels carrying fluid to essential tissues and areas for recirculation or excretion. When vessels become damaged severed or occluded, certain sections may be bypassed to allow for the free flow of fluids, and an anastomosis can be performed. Anastomosis is a procedure to connect healthy sections of two structures, organs, or spaces in the body as part of a bypass procedure or after diseased tissue has been surgically removed. It commonly refers to a connection that is created between two tubular structures, such as a transected blood vessel, to optimize or redirect flow. Examples of surgical anastomoses include a colostomy with an opening created between the bowel and the abdominal skin, and the arterio-venous fistula with an opening created between an artery and vein for hemodialysis.

Vascular anatomosis, which joins two ends of a transected blood vessel with connectors such as sutures or staples, may be used to reroute blood flow around an occlusion or area of stenosis. This procedure has been used to revascularize the tissue downstream of an arterial blockage by employing bypass grafting of artificial, in-situ venous, or transplanted venous grafts. In the past, bypass grafting has required extensive surgery.

For example, when performing traditional coronary artery bypass grafting (CABG) procedures, anastomosis often requires the heart to be manipulated, isolated from the systemic circulation, and stopped for an extended period of time, so that the anastomosis site on the heart is blood-free and still during suturing. Risks of post-surgical complications increase the longer the heart is under cardioplegic arrest.

Stapling and coupling procedures are challenging for vascular anastomosis, which requires that stapling instruments and staples conform to smaller sized vessels. Often stapling or coupling devices require the everting of the vessel walls, which may not always be practical, especially for small arteries that are more prone to tear. Furthermore, the triggering force of stapling devices and incorrect spacing between staple points have the potential of vessel laceration or leakage from the anastomosis.

A common procedure for performing the anastomosis during bypass surgery requires the use of very small sutures, loupes and microsurgical techniques for connecting the vessels together. Suturing the anastomosis is time-consuming and may not provide a leak-free seal. In some cases the vessel wall may not be strong enough for suturing a bypass. Consequently, medical professionals and surgeons continue to need better techniques to protect tissue, as well as reduce the laborious and time-consuming task of vessel suturing.

One method available for expediting anastomosis procedures is through the use of anastomosis fittings and devices for joining blood vessels together. Whenever foreign material is introduced to the blood flow path, there may be greater possibilities of hemolysis and thrombosis at the grafts. Other problems include stenosis at the anastomosis and intracardiac hemorrhages from ruptured venules. It is critical for the materials of these fittings and devices to be biocompatible, to have burst strengths equivalent to sutures, to create quick hemostasis, and to protect surrounding tissue. The area where the anastomosis is created also may benefit from localized drug delivery.

Stents have been used to secure a graft vessel to a target vessel during a surgical procedure, one being described in “Method and System for Attaching a Graft to a Blood Vessel”, Yencho et al., U.S. Pat. No. 6,497,710 issued Dec. 24, 2002. The anastomotic stent is used in a sutureless vascular anastomosis, such as may be used in coronary artery bypass grafting (CABG).

An area of growing interest has been the performance of minimally invasive approaches for performing coronary artery bypasses and other anastomosis procedures. The results from recent clinical research that use minimally invasive approaches hold promise for early graft patency, lower rates of morbidity and mortality, and shortened hospital stays. When compared with conventional surgical approaches, minimally invasive coronary artery bypasses can result in a lower need of intraoperative and postoperative blood transfusions.

Included among the newer interventional techniques are percutaneous, transluminal techniques for bypassing obstructions in coronary or other arteries through the use of one or more adjacent veins as in-situ bridge or bypass conduits. Catheters are used to perform extra-luminal procedures outside the diseased vessel lumen. In some instances, these procedures may be performed by a venous approach wherein a flexible, tissue-penetrating catheter is inserted and advanced into a first blood vessel vein to a desired location, and a tissue penetrator penetrates from the first vessel into a target location such as the lumen of an adjacent second vessel. The desired passageway or puncture is formed initially by facilitating the passage of a tissue penetrator from a catheter, through the wall of the vein in which the catheter is positioned, and into a target location. One such catheter is described by Flaherty and others in “Tissue Penetrating Catheters having Integral Imaging Transducers and their Methods of Use”, U.S. Pat. No. 6,375,615 issued Apr. 23, 2002. The tissue penetrator may be a flow of energy such as a laser, or an elongated penetration member. Some of the tissue penetrating catheters have a penetrator direction marker to indicate the direction in which the tissue penetrator passes from the catheter, which then may be detected with an ultrasound imaging device that is advanced through another transvascular catheter.

Currently, a variety of catheter-deployed stents are used within arteries to treat stenoses, strictures, aneurysms, and the like. For example, a stent may be implanted within a partially occluded region of an artery to retain stenotic material beneath the stent or to open the lumen of the artery for improving blood flow therethrough.

One example of a connector device that is deployed intravascularly with the aid of a catheter is disclosed in “Devices for Forming and/or Maintaining Connections between Adjacent Anatomical Conduits”, Kim et al., U.S. patent application Publication 20020029079 published Mar. 7, 2002. The device generally comprises two or more radially expandable annular members that have one or more elongated strut members extending between those members. Another method and apparatus for using the vascular system as a conduit to reach other vascular and extravascular locations is disclosed by Makower in “Devices for Connecting Anatomical Conduits such as Vascular Structures”, U.S. Pat. No. 6,231,587 issued May 15, 2001. A catheter-deployed connecting segment or stent provides a conduit from a first vessel, through the vessel wall, and then through a wall of and into an adjacent vessel.

A segmented, implantable device for interconnecting vessels in a minimally invasive manner is disclosed by Shennib and others in “Device for Interconnecting Vessels in a Patient”, U.S. Pat. No. 6,464,709 issued Oct. 15, 2002; “Devices and Methods for Interconnecting Vessels”, U.S. Pat. No. 6,458,140 issued Oct. 1, 2002; “Device for Interconnecting Vessels in a Patient”, U.S. Pat. No. 6,251,116 issued Jun. 26, 2001, and “Method for Interconnecting Vessels in a Patient”, U.S. Pat. No. 6,165,185 issued Dec. 26, 2000. The device, which is deployed by a specially designed catheter, comprises a first bendable segment, a second bendable segment, and a flow opening along the periphery of the two connected segments. The segments subsequently conform to the interior walls of a vessel to provide a sealing contact along the contact surface of the segment inserted within the vessel. The two vessels are required to be immediately adjacent and there is no stent framework for reinforcing the interior walls of the vessels near the point of interconnection.

Research efforts are focusing on creating stents and bypass conduit structures that can effectively engage curved vessel regions and passed-through penetration regions. The structure of many currently available stents may substantially resist bending and may be unable to conform to a curved portion of a vessel. For example, when the stent is being delivered along a circuitous arterial path, the stiffness of the stent, particularly in its contracted condition, may impair advancement of the stent around tight curves in the lumen. An example of a stent that is malleable is disclosed in “Deformable Scaffolding Multicellular Stent”, U.S. patent application No. 20020111672, Kim et al., published Aug. 15, 2002. The plastically deformable stent has cylindrical segments with connectors extending between adjacent segments. The connectors are designed to support the vessel wall to maintain a desired open lumen cross-section clear of exposed material extending from the vessel wall into the bloodstream.

Besides needing to be flexible, a stent, conduit or connector device that is being used to bypass an occlusion needs to maintain fluidic connection between the openings that are formed in adjacent vessels. This can be difficult particularly when the two vessels are not in direct contact with one another. One device using a tubular member between the two vessels is described in “Anastomosis Device and Method”, Duhaylongsod et al., U.S. Pat. No. 6,241,741 issued Jun. 5, 2001. A device with a fastener is inserted from one vessel at least part way into a second vessel, with an end of the fastener dissecting and extending through the second vessel and at least partially expanding radially for securing the first vessel to an inner wall of the second vessel. The second vessel has an opening formed in a sidewall for insertion of the device. The device includes a radially expandable tubular member that is preformed with a bend along its central longitudinal axis. A portion of the tubular member extends out from the sidewall of the second vessel while an end portion of the tubular member extends coaxially with the second vessel when the tubular member is inserted in the second vessel. This type of device, however, provides minimal protection from the tubular member against further dissection, tearing and weakening of the vessel wall at the point of egression.

Therefore, improved devices, methods and systems are desirable to reduce the difficulty of creating the vascular anastomosis and to provide a reliable anastomosis between two vessels. The devices that are used need to be biocompatible, and their connections, sections and conduits need to be secured effectively among each other, as well as to the vessels. Improved techniques for anastomosis that both provide greater tissue protection and require less time are also beneficial. Accordingly, there is a need for improved devices and methods for interconnecting vessels within a body with a maneuverable and flexible but secure configuration, in furtherance of a minimally invasive, therapeutic purpose.

### SUMMARY OF THE INVENTION

One aspect of the present invention provides an anastomotic apparatus for a bypass. The anastomotic apparatus includes a first anastomotic stent with a side port, a bridge conduit with a first end and a second end, and a second anastomotic stent with a side port. The bridge conduit includes a first and a second end, the first end in communication with the side port of the first anastomotic stent and the second end in communication with the side port of the second anastomotic stent. The first anastomotic stent is positionable in a first vessel and the second anastomotic stent is positionable in a second vessel. The bridge conduit provides a pathway for fluid flow between the first and second vessels

Another aspect of the present invention is a method of bypassing an occlusion in a vessel. A first anastomotic stent with a side port is deployed in a first vessel adjacent to an occlusion in the first vessel, and a second anastomotic stent with a side port is deployed in a second vessel. Openings aligned with the side ports are formed in the first and second vessels. A bridge conduit is deployed through the vessel openings and coupled to the side ports of the anastomotic stents. Fluid flows through the bridge conduit between the first and second vessels.

Another aspect of the present invention is an anastomotic stent for a bypass including a stent framework with a generally tubular structure and a side port between first and second ends of the anastomotic stent. The side port is endovascularly connectable to a bridge conduit with a coupling mechanism to provide a pathway for fluid flow through the side port and at least one end of the stent framework.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiment of the present invention are illustrated by the accompanying figures, wherein:

FIG. 1 is an illustration of an anastomotic stent coupled to a catheter, in accordance with one embodiment of the current invention;

FIG. 2 is an illustration of an anastomotic apparatus, in accordance with one embodiment of the current invention;

FIG. 3 is an illustration of an anastomotic stent and a bridge conduit, in accordance with one embodiment of the current invention;

FIG. 4 is an illustration of an anastomotic-stent and a bridge conduit, in accordance with another embodiment of the current invention;

FIG. 5 is an illustration of a coronary bypass using an anastomotic apparatus, in accordance with one embodiment of the current invention;

FIG. 6 is an illustration of a femoral bypass using an anastomotic apparatus, in accordance with one embodiment of the current invention; and

FIG. 7 is a flow diagram of a method for bypassing an occlusion in a vessel, in accordance with one embodiment of the current invention.

### DETAILED DESCRIPTION OF THE PRESENTLY PREFERRED EMBODIMENTS

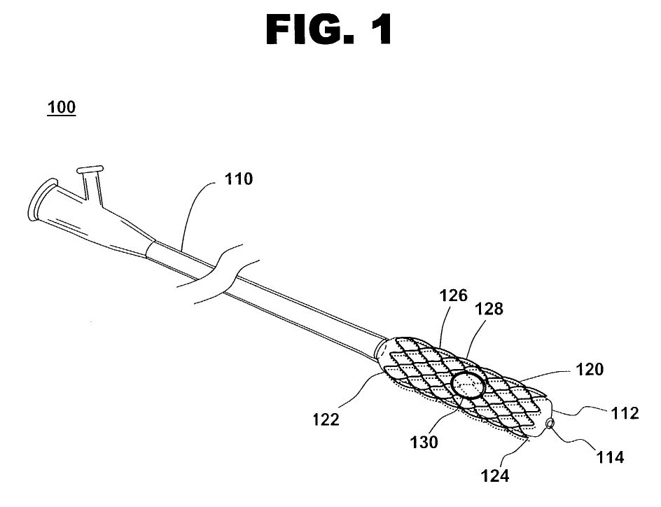

FIG. 1 shows an illustration of an anastomotic stent coupled to a catheter, in accordance with one embodiment of the present invention at 100. Anastomotic stent 120, also referred to as a port stent, has a first end 122 and a second end 124, with a side port 130 between first end 122 and second end 124. Anastomotic stent 120 includes a stent framework 126 having a generally tubular structure. Side port 130 is endovascularly connectable to a bridge conduit, sometimes referred to as a graft or a bypass conduit, with a coupling mechanism to provide a pathway for fluid flow through side port 130 and at least one end 122, 124 of stent framework 126. When deployed in a vessel, first end 122 and second end 124 of anastomotic stent 120 inhibit further dissection of the vessel walls around side port 130, reduce trauma to the vessel walls, and provide mechanical support for the walls of the vessel in the vicinity of the connection between the graft or bridge conduit and side port 130 of anastomotic stent 120. In cases where the vessel wall is not strong enough for suturing a bypass, anastomotic stent 120 with side port 130 provides additional strength to connect the graft or bridge conduit.

An anastomotic apparatus including one or more anastomotic stents 120 may help treat, for example, heart disease, various cardiovascular ailments, and other vascular conditions by using catheter-deployed anastomotic stents. Anastomotic stent 120 may be used to bypass, for example, one or more blockages, occlusions, stenoses, or diseased regions in a coronary artery, femoral artery, peripheral arteries, and other arteries in the body. Treatment of vascular conditions may include the prevention or correction of various ailments and deficiencies associated with the cardiovascular system, the cerebrovascular system, urinogenital systems, biliary conduits, abdominal passageways and other biological vessels within the body.

Anastomotic stent 120 may be coupled to a delivery catheter 110. Anastomotic stent 120 may be deployable with a balloon 112, the balloon positioned between anastomotic stent 120 and delivery catheter 110. Delivery catheter 110, which is used to position anastomotic stent 120 in a vessel of a body, is typically inserted through a small incision of the leg and into the femoral artery, and directed through the vascular system to a desired place in the vessel. Guide wires threaded through an inner member 114 of delivery catheter 110 assist in positioning and orienting anastomotic stent 120. The position of anastomotic stent 120 may be monitored, for example, with a fluoroscopic imaging system or an x-ray viewing system and radiopaque markers on anastomotic stent 120, radiopaque markers on delivery catheter 110, or contrast fluid injected into an inner lumen of delivery catheter 110 and into balloon 112. Inflation of balloon 112 with an external source of pressure enlarges anastomotic stent 120, plastically deforming stent framework 126 until the desired diameter is obtained and anastomotic stent 120 is secured in the vessel. Release of pressure deflates balloon 112, separating balloon 112 from anastomotic stent 120 prior to removal of balloon 112 and delivery catheter 110. Alternatively, delivery catheter 110 may comprise a delivery sheath coupled to delivery catheter 110 that retracts to deploy a self-expanding version of anastomotic stent 120. The delivery sheath surrounds a self-expanding version of anastomotic stent 120, which is torn away or retracted to allow deployment of anastomotic stent 120. Delivery catheters 110 with inflatable balloons 112 and retractable sheaths are well known in the art.

A drug-polymer coating 128 may be disposed on at least a portion of stent framework 126. Drug-polymer coating 128 may comprise one or more polymeric materials suitable for coating anastomotic stent 120 and for deployment within the body. Drug-polymer coating 128 may comprise a biodegradable polymer or a biostable polymer. Drug-polymer coating 128 may comprise, for example, a biodegradable polymer such as polycaprolactone (PCL), polyglycolide (PGA) or poly(lactide-co-glycolide) (PLGA), or a biostable polymer such as a silicone-urethane copolymer, a polyurethane, or ethylene vinyl acetate (EVA).

Drug-polymer coating 128 may include or encapsulate one or more therapeutic agents. Drug-polymer coating 128 may comprise one or more therapeutic agents dispersed within or encased by a polymeric coating, which are eluted from anastomotic stent 120 with controlled time delivery after deployment of anastomotic stent 120 within a body. A therapeutic agent is capable of producing a beneficial effect against one or more conditions including coronary restenosis, cardiovascular restenosis, angiographic restenosis, arteriosclerosis, hyperplasia, and other diseases and conditions. For example, the therapeutic agent can be selected to inhibit or prevent vascular restenosis, a condition corresponding to a narrowing or constriction of the diameter of the bodily lumen where the stent is placed. Drug-polymer coating 128 may comprise, for example, an antirestenotic agent such as rapamycin, a rapamycin analogue, or a rapamycin derivative to prevent or reduce the recurrence of narrowing and blockage of the bodily vessel. Drug-polymer coating 128 may comprise an antisense agent, an antineoplastic agent, an antiproliferative agent, an antithrombogenic agent, an anticoagulant, an antiplatelet agent, an antibiotic, an anti-inflammatory agent, a steroid, a gene therapy agent, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, a therapeutic substance, or a combination thereof. The elution rates of the therapeutic agents into the body and the tissue bed surrounding the stent framework are based on the constituency and thickness of drug-polymer coating 15 128, the nature and concentration of the therapeutic agents, the thickness and composition of any cap or barrier coats with the coating, and other factors. Drug-polymer coating 128 may include and elute multiple therapeutic agents. Drug-polymer coating 128 can be tailored to control the elution of one or more therapeutic agents primarily by diffusion processes. In some cases, a portion of the polymeric coating is absorbed into the body to release therapeutic agents from within the coating.

Incorporation of a drug or other therapeutic agent into drug-polymer coating 128 allows, for example, the rapid delivery of a pharmacologically active drug or bioactive agent within twenty-four hours of surgery, with a slower, steady delivery of a second bioactive agent over the next three to six months. For example, a first therapeutic agent may comprise an antirestenotic drug such as rapamycin, a rapamycin analogue, or a rapamycin derivative. The second therapeutic agent may comprise an anti-inflammatory drug such as dexamethosone.

An adhesion layer may be positioned between drug-polymer coating 128 and stent framework 126 to improve the adhesion of the drug-polymer coating and its durability. The adhesion layer may be a polymeric material or any material that adheres well to the underlying stent framework, particularly a metallic base of anastomotic stent 120. The adhesion layer is selected to adhere well to anastomotic stent 120 and to be readily coated with another polymeric material such as drug-polymer coating 128. The adhesion layer may be any suitable adhesion layer material such as parylene, polyurethane, phenoxy, epoxy, polyimide, polysulfone, or pellathane.

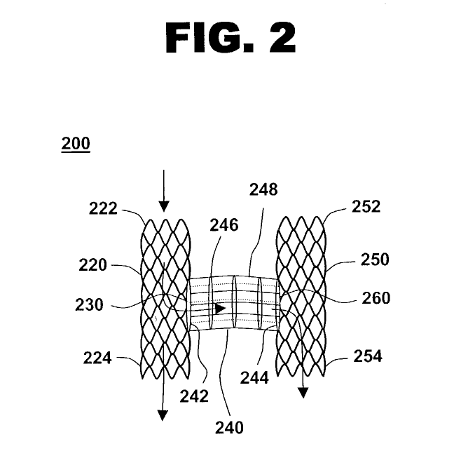

FIG. 2 shows an illustration of an anastomotic apparatus, in accordance with one embodiment of the present invention at 200. Anastomotic apparatus 200 includes a first anastomotic stent 220 with a side port 230, a graft or bridge conduit 240, and a second anastomotic stent 250 with a side port 260. Bridge conduit 240 includes a first end 242 in communication with side port 230 of first anastomotic stent 220, and a second end 244 in communication with side port 260 of second anastomotic stent 250. First anastomotic stent 220 is positionable in a first vessel and second anastomotic stent 250 is positionable in a second vessel. Bridge conduit 240 provides a pathway for fluid flow between the first and second vessels. Generally, anastomotic apparatus 200 forms an H-shape when deployed in a body, with bridge conduit 240 forming a bridge between the first vessel and the second vessel.

First anastomotic stent 220 includes a first end 222 and a second end 224. Anastomotic apparatus 200 allows a fluid to flow into first end 222, with at least a portion of the fluid flowing out of side port 230 into bridge conduit 240 and the remaining portion of the fluid flowing out of second end 224. Similarly, anastomotic apparatus 200 allows a fluid to flow through bridge conduit 240 into side port 230, with at least a portion of the fluid flowing out through first end 222 and the remaining portion of the fluid, if any, flowing out of second end 224. Generally, first end 222 and second end 224 of first anastomotic stent 220 are fluidically interchangeable. First end 222 and second end 224 of first anastomotic stent 220 may include, for example, one or more rows of struts on each side of side port 230. In the vicinity of side port 230, the stent struts comprise, for example, a set of axially elongated struts that curve around the periphery of side port 230 to allow mating with bridge conduit 240.

Bridge conduit 240 fluidly couples side port 230 of first anastomotic stent 220 to side port 260 of second anastomotic stent 250. Fluid entering, for example, a first end 222 of first anastomotic stent 220 would be rerouted through side port 230 and through bridge conduit 240 into second anastomotic stent 250, then flowed out of a first end 252 or a second end 254 of second anastomotic stent 250 or both, depending on any existing or added restrictions in the second vessel. In another example, fluid entering either first end 252 or second end 254 of second anastomotic stent 250 flows through bridge conduit 240 and out of first end 222 or second end 224 of first anastomotic stent 220.

Bridge conduit 240 includes a conduit framework 246 and a polymeric conduit wall 248 connected to at least a portion of conduit framework 246. First end 242 of bridge conduit 240 mates, for example, with side port 230 of first anastomotic stent 220. Conduit framework 246 may comprise, for example, one or more axially directed struts between first end 242 and second end 244 of bridge conduit 240 with conduit wall 248 attached to the one or more axially directed struts. Conduit framework 246 may comprise, for example, a series of sinusoidal-shaped struts forming a generally tubular shape that allow conduit framework 246 to be radially expanded. Conduit framework 246, a first stent framework 226 of first anastomotic stent 220, and a second stent framework 256 of second anastomotic stent 250 may have a metallic or a polymeric base, comprising a material such as stainless steel, nitinol, tantalum, MP35N alloy, platinum, titanium, a chromium-based alloy, a cobalt-based alloy, a suitable biocompatible alloy, a suitable biocompatible material, a biocompatible polymer, or a combination thereof. A drug-polymer coating may be disposed on at least a portion of conduit framework 246, first stent framework 226, and second stent framework 256.

Conduit wall 248 may comprise biocompatible wall and membrane materials typically used for grafts such as polytetrafluoroethylene (PTFE), expanded polytetrafluoroethylene (e-PTFE), polyurethane, or polyester, with sufficient strength and pliability to withstand pressure generated during blood flow. In some cases, the polymeric coverings may be used for suturing. Conduit wall 248 may be, for example, porous to permit endothelialization. Conduit wall 248, for example, may be largely positioned inside of conduit framework 246, wrapping over conduit framework near first end 242 and second end 244. Conduit wall 248 may comprise, for example, wires or strands of radiopaque material such as platinum, palladium, tantalum or gold between polymeric layers or woven into the wall fabric to highlight the covering during positioning and deployment. Conduit wall 248 may suspend or encapsulate, for example, medications that prepare the vessel wall for bridge conduit 240 such as medications that work to thicken the arterial walls in the vicinity of side port 230 or medications that prevent the vessel from rejecting the graft. First anastomotic stent 220 and second anastomotic stent 250 may be covered in part with a polymeric covering.

Bridge conduit 240 is deployable with a balloon catheter or a delivery sheath coupled to a catheter that retracts to deploy bridge conduit 240. For example, inflating a balloon coupled between bridge conduit 240 and a catheter may deploy bridge conduit 240. In another example, retracting a delivery sheath coupled to a catheter may deploy a self-expanding bridge conduit 240. In another example, a delivery catheter with a three-section balloon or multiple balloons are used to deploy first anastomotic stent 220, bridge conduit 240, and second anastomotic stent 250.

Bridge conduit 240 may be coupled to side port 230 of first anastomotic stent 220 or to side port 260 of second anastomotic stent 250 during deployment of bridge conduit 240. Bridge conduit may be coupled to side ports 230 and 260 with a coupling maneuver such as flaring, clasping, locking, engaging, suturing, or securing. For example, self-expansion of bridge conduit 240 may couple bridge conduit 240 to side ports 230 and 260. In another example, a first end 242 of bridge conduit 240 may be inserted through side port 230 of first anastomotic stent 220 and flared by an inflatable balloon to lock and secure bridge conduit 240 to side port 230. In another example, one or more clasps on first end 242 of bridge conduit 240 or on side port 230 of first anastomotic stent 220 may be engaged when bridge conduit 240 is deployed to engage and secure bridge conduit 240 to side port 230. In another example, a locking mechanism formed in either bridge conduit 240 or side port 230 or both may be locked when bridge conduit 240 is deployed to secure bridge conduit 240 to side port 230. Other coupling mechanisms on bridge conduit 240, side port 230, or both may be used to connect and couple bridge conduit 240 to side port 230 of first anastomotic stent 220 during an endovascular maneuver directed by catheters, guide wires, and other endovascular tools. Friction fitting or other coupling mechanisms such as flanges, snaps, tabs, slots, and barbs may be used to connect and secure bridge conduit 240 to side port 230 of first anastomotic stent 220. Bridge conduit 240 may be connected to side port 230, for example, with staples that are applied using an endovascular stapling tool. In another example, a plurality of eyelets may be pre-formed at first end 242 of bridge conduit 240 and around the periphery of side port 230, and used to connect bridge conduit 240 to side port 230 by suturing or stapling through the eyelets.

Similar coupling mechanisms and techniques may be used on second end 244 of bridge conduit 240 for coupling bridge conduit 240 to side port 260 of second anastomotic stent 250. One or more balloons may be used to enlarge and deploy bridge conduit 240 so that first end 242 of bridge conduit 240 is in communication with side port 230 of first anastomotic stent 220, and that second end 244 of bridge conduit 240 is in communication with side port 260 of second anastomotic stent 250, resulting in a pathway for fluid flow between the first vessel and the second vessel.

In another embodiment, an anastomotic apparatus includes a first anastomotic stent 220 with a first end 222 and a bridge conduit 240 with a first end 242 coupled to a side port 230 of first anastomotic stent 220 and a second end 244 coupled to a side port 260 of a second anastomotic stent 250. Anastomotic stents 220 and 250 and bridge conduit 240 are deployable from a single catheter. Second end 224 of first anastomotic stent 220 and first end 252 of second anastomotic stent 250 are partially or fully capped, or omitted altogether from the apparatus.

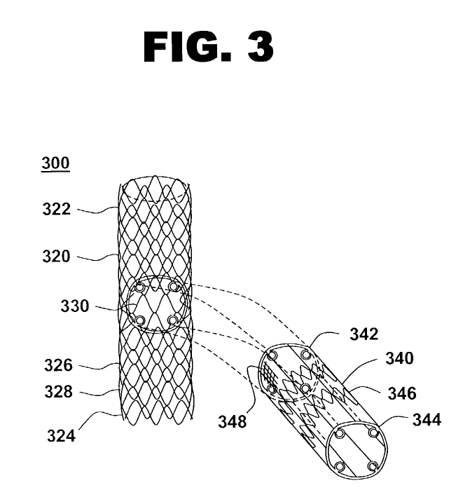

FIG. 3 shows an illustration of anastomotic stent coupled to a bridge conduit, in accordance with one embodiment of the present invention at 300. Anastomotic apparatus 300 includes a first anastomotic stent 320 and a bridge conduit 340. First anastomotic stent 320 includes a first end 322 and a second end 324 with a side port 330 between first end 322 and second end 324. First anastomotic stent 320 includes a stent framework 326 and may have a drug-polymer coating 328 disposed on stent framework 326. Bridge conduit 340 has a first end 342 and a second end 344, and includes a conduit framework 346 with a conduit wall 348. A drug-polymer coating may be disposed on conduit framework 346.

First end 342 of bridge conduit 340 is coupled to side port 330 of first anastomotic stent 320. In this example, a set of eyelets are formed on conduit framework 346 near first end 342 of bridge conduit 340 and on stent framework 326 near side port 330 of first anastomotic stent 320. The eyelets may be used, for example, to suture or staple first end 342 of bridge conduit 340 to side port 330 of first anastomotic stent 320 prior to, during, or after deployment of bridge conduit 340 so that first end 342 of bridge conduit 340 is in communication with side port 330 of first anastomotic stent 320.

Second end 344 of bridge conduit 340 is coupled to a side port of a second anastomotic stent, not shown for purposes of clarity.

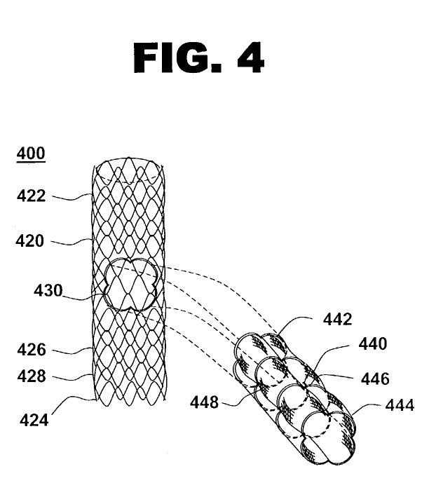

FIG. 4 shows an illustration of an anastomotic stent and a bridge conduit, in accordance with another embodiment of the present invention at 400. Anastomotic apparatus 400 includes a first anastomotic stent 420 and a bridge conduit 440. First anastomotic stent 420 includes a first end 422 and a second end 424 with a side port.430 between first end 422 and second end 424. First anastomotic stent 420 includes a stent framework 426 and may have a drug-polymer coating 428 disposed on stent framework 426. Bridge conduit 440 has a first end 442 and a second end 444, and includes a conduit framework 446 with a conduit wall 448. A drug-polymer coating may be disposed on conduit framework 446.

First end 442 of bridge conduit 440 is coupled to side port 430 of first anastomotic stent 420. In this example, a press fit or a friction fit is used to couple bridge conduit 440 to side port 430 of first anastomotic stent 420 so that first end 442 of bridge conduit 440 is in communication with side port 430 of first anastomotic stent 420. The friction fit may be achieved during deployment of bridge conduit 440, such as when bridge conduit 440 is allowed to self-expand or is enlarged by a balloon to affix first end 442 of bridge conduit 440 to stent framework 326 of first anastomotic stent 420. Similarly, a friction fit may be achieved during deployment of bridge conduit 440 to affix second end 444 of bridge conduit 440 to a side port of a second anastomotic stent, not shown for clarity. In one example, axially elongated struts in the vicinity of side port 430 of first anastomotic stent 420 are bent into a curved shape when bridge conduit 440 is expanded or enlarged.

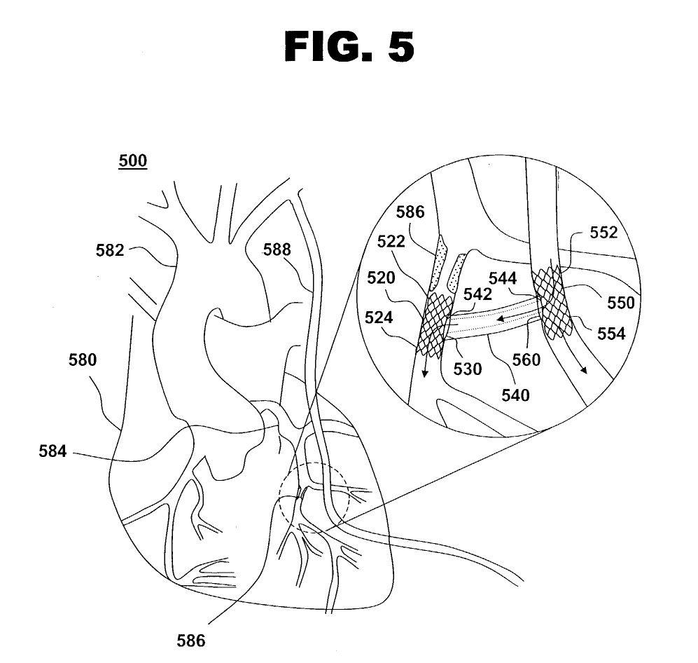

FIG. 5 shows an illustration of a coronary bypass using an anastomotic apparatus, in accordance with one embodiment of the present invention at 500. Coronary bypass 500, herein referred to as a minimally-invasive coronary artery bypass graft (mini-CABG) for a heart 580, includes a first anastomotic stent 520 with a side port 530 in a first vessel, a graft or bridge conduit 540 including a first end 542 and a second end 544, and a second anastomotic stent 550 including a side port 560 in a second vessel. Each element of the anastomotic apparatus is catheter-deployable. In this implementation, a coronary artery 584 is partially or fully blocked by one or more stenoses or occlusions 586 that may prevent adequate flow of blood to extremities of heart 580 from aorta 582. Internal thoracic-artery 588 connected between aorta 582 and vasculature in the chest area, is partially or fully redirected to provide additional flow of oxygenated blood from aorta 582 to portions of coronary artery 584 at locations downstream of occlusion 586. The mini-CABG is performed using minimally-invasive endovascular deployment of an anastomotic assembly, part of which is deployed in coronary artery 584 and another part which is deployed in internal thoracic artery 588 or other suitable artery or vasculature near occlusion 586.

The anastomotic apparatus includes a first anastomotic stent 520 including a side port 530 positioned and deployed in coronary artery 584 with a first end 522 adjacent to occlusion 586, a second anastomotic stent 550 including a side port 560 positioned in internal thoracic artery 588, and a bridge conduit 540 including a first end 542 and a second end 544. First end 542 of bridge conduit 540 is in communication with side port 530 of first anastomotic stent 520, and second end 544 of bridge conduit 540 is in communication with side port 560 of second anastomotic stent 550. Bridge conduit 540 provides a pathway for fluid flow between aorta 582 and coronary artery 584 via internal thoracic artery 588, bypassing occlusion 586 in coronary artery 584. In this example, a portion of fluid flowing into first end 552 of second anastomotic stent 550 may flow out of a second end 554 into capillaries in the chest cavity, so that at least a portion of the pre-bypass blood flow may reach their initial destination. The portion of blood directed into bridge conduit 540 that enters first end 552 of second anastomotic stent 550 may be controlled by full or partial closure of second end 554 of second anastomotic stent 550 as desired, or by intentionally blocking part or all of internal thoracic artery 588 downstream of first anastomotic stent 520.

The anastomotic apparatus allows oxygenated blood to flow through first end 552 of second anastomotic stent 550, through side port 560 and bridge conduit 540, through side port 530 of first anastomotic stent 520, and out a second end 524 of first anastomotic stent 520.

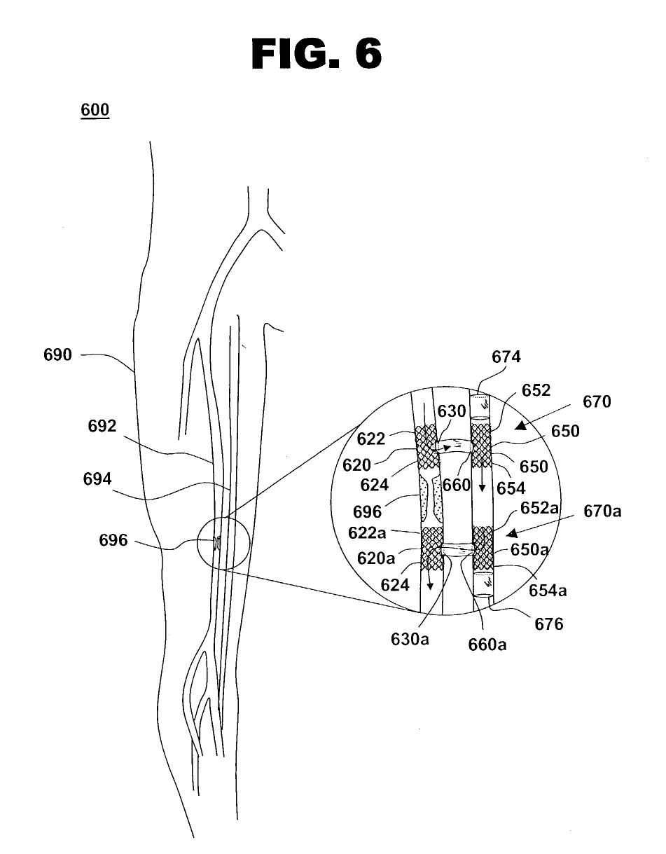

FIG. 6 shows an illustration of a femoral bypass using an anastomotic apparatus, in accordance with one embodiment of the present invention at 600. Femoral bypass 600 includes a first anastomotic apparatus 670 and a second anastomotic apparatus 670a cooperating to bypass a partial or full occlusion 696 in a femoral artery 692 of a leg 690 using part of a saphenous vein 694. The bypass may be applied to other peripheral arteries in the leg, such as the deep femoral artery, the superficial femoral artery, the popliteal artery, the anterior tibial artery, the posterior tibial artery, or the peroneal artery. For example, a first anastomotic stent 620 of a first anastomotic apparatus 670 is placed in the popliteal artery with a second anastomotic stent 650 of first anastomotic apparatus 670 placed in saphenous vein 694, and a third anastomotic stent 620a of a second anastomotic apparatus 670a is placed in the posterior tibial artery with a second anastomotic stent 650a of second anastomotic apparatus 670a placed further down the leg in saphenous vein 694.

First anastomotic apparatus 670 includes a first anastomotic stent 620 including a side port 630, a bridge conduit 640 including a first end 642 and a second end 644, and a second anastomotic stent 650 including a side port 660. First anastomotic stent 620 is positioned and deployed in femoral artery 692 with a second end 624 adjacent to occlusion 696 in femoral artery 692 and a first end 622 positioned upstream from second end 624. Second anastomotic stent 650 is positioned and deployed in saphenous vein 694, with a first end 652 nearer the heart than a second end 654. First end 642 of bridge conduit 640 is in communication with side port 630 of first anastomotic stent 620, and second end 644 of bridge conduit 640 is in communication with side port 660 of second anastomotic stent 650. Bridge conduit 640 provides a pathway for fluid flow between femoral artery 692 and saphenous vein 694.

Second anastomotic apparatus 670a includes a third anastomotic stent 620a including a side port 630a, a bridge conduit 640a including a first end 642a and a second end 644a, and a fourth anastomotic stent 650a including a side port 660a. Third anastomotic stent 620a is positioned and deployed in femoral artery 692 with a first end 622a adjacent to occlusion 696 opposite first anastomotic stent 620 and a second end 624a positioned downstream from first end 622a. Fourth anastomotic stent 650a is positioned and deployed in saphenous vein 694, with a first end 652a downstream of a second end 654a. First end 642a of bridge conduit 640a is in communication with side port 630a of third anastomotic stent 620a, and second end 644a of bridge conduit 640a is in communication with side port 660a of fourth anastomotic stent 650a. Bridge conduit 640a provides a pathway for fluid flow between saphenous vein 694 and femoral artery 692. In this example, blood flow from femoral artery 692 is directed from first end 622 of first anastomotic stent 620, through side port 630 and bridge conduit 640, into side port 660 of second anastomotic stent 650 and out through second end 654 of second anastomotic stent 650, and through a section of saphenous vein 694 towards second anastomotic apparatus 670a in a direction opposite of normal blood flow through the vein. The blood flow enters first end 652a and out through side port 660a of fourth anastomotic stent 650a, through bridge conduit 640a and side port 630a of third anastomotic stent 620a, and continues down leg 690 towards the lower extremities of the leg and foot, thereby bypassing occlusion 696 in femoral artery 692. First anastomotic apparatus 670 allows a portion of the blood from femoral artery 692 to travel through first anastomotic stent 620, out second end 624 of first anastomotic stent 620, and through occlusion 696 as occlusion 696 allows, such as in the case of a partial occlusion.

In this example, blood flow traverses a direction in saphenous vein 694 opposite to normal blood flow. Vascular plugs 674 and 676 may be inserted, deployed, or otherwise employed in saphenous vein 694 to block normal blood flow back to the heart and to aid in the transport of blood through the anastomotic stents and bridge conduits. Tissue valves incumbent within saphenous vein 694 may need to be disfunctionalized by stenting or other procedures to avoid inadvertent blockage of blood flow opposite to normal blood flow. Alternatively, saphenous vein 694 may be clipped or stapled to partially or fully block flow in unintended directions.

In another embodiment of the femoral bypass, a portion of the femoral vein in the leg 690 is used in lieu of part of the saphenous vein 694.

Although the femoral artery is bypassed in this example, other arteries and vasculature in the body may be suitably bypassed. In one example, the first anastomotic stent is positioned in an arterial vessel and the second anastomotic stent is positioned in a venous vessel. In another example, the first anastomotic stent is positioned in a first arterial vessel and the second anastomotic stent is positioned in a nearby arterial vessel. In another example, the first anastomotic stent is positioned near a blockage or occlusion in a vessel and the second anastomotic stent is positioned in the same vessel opposite the occlusion, the bridge conduit or graft connecting the side ports of the upstream and downstream anastomotic stents.

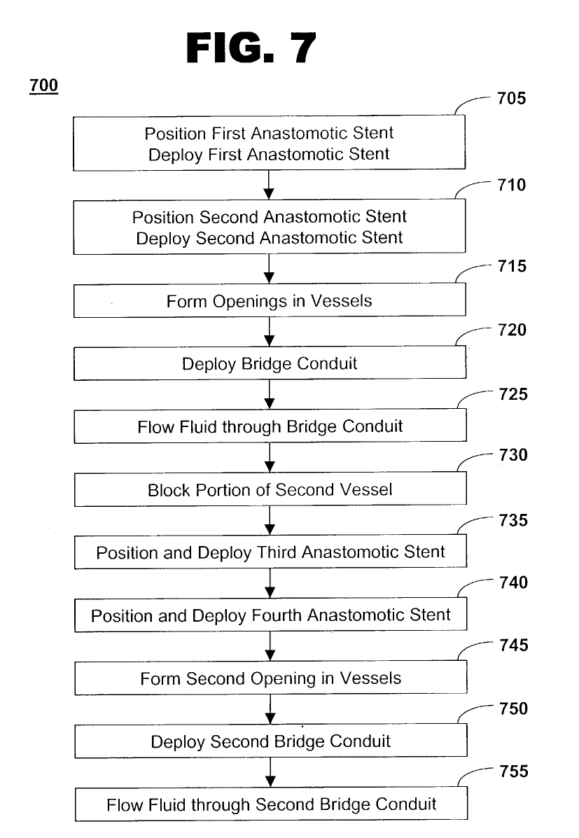

FIG. 7 shows a flow diagram of a method for bypassing an occlusion in a vessel, in accordance with one embodiment of the present invention at 700. Bypass method 700 includes various steps for redirecting flow from one vessel to another, and in some cases, back again using largely endovascular procedures, tooling, and techniques.

A first anastomotic stent is positioned and deployed in a first vessel, as seen at block 705. The first anastomotic stent is placed adjacent to an occlusion in the first vessel. In some cases, the anastomotic stent is deployed over the stenosis or occlusion. Balloon catheters and delivery catheters with retractable sheaths may be used to deploy the anastomotic stent. For example, a balloon coupled between the anastomotic stent and a catheter is inflated to deploy the anastomotic stent. In another example, the anastomotic stent includes a retractable sheath coupled to a catheter that is retracted to allow expansion of a self-expanding version of the anastomotic stent. The anastomotic stent has a side port, which is oriented in a preferred direction during the deployment of the anastomotic stent. The anastomotic stent may be positioned and deployed in a vein such as the saphenous vein, an artery such as the coronary artery, internal thoracic artery, or femoral artery, or other vasculature within the body.

A second anastomotic stent is positioned and deployed in a second vessel, as seen at block 710. The second anastomotic stent is generally placed in a suitable vessel near the first vessel to minimize the bypass distance. A balloon catheter may be used to deploy the second anastomotic stent, using a guide wire for positioning and an externally applied pressure for inflating the balloon. A second self-expanding anastomotic stent may be deployed using a delivery catheter with a retractable sheath. The second anastomotic stent includes a side port that is generally oriented in the direction of the side port of the first anastomotic stent.

Openings aligned with the side ports are formed in the first and second vessels, as seen at block 715. The openings are formed in the vessel wall adjacent to the side ports, and may be formed from within the vessel with, for example, catheter-based cutting tools, endoscopic cutting lasers, knives or scissors.

A bridge conduit is deployed through the vessel openings, as seen at block 720. The bridge conduit may be positioned, for example, by first coupling two guide wires together, one from each vessel, then pushing the bridge conduit along one of the guide wires until the bridge conduit is suitably positioned between the side ports of the anastomotic stents. Small loops or magnets in the tips of the guide wires may be used, for example, to couple or connect the tip of one guide wire to the tip of another guide wire. In another example, the bridge conduit may be positioned by first maneuvering a guide wire through the side port of the first anastomotic stent, through intermediary biological material, and through the side port of the second anastomotic stent, then pushing the bridge conduit along the guide wire until the conduit is suitably positioned and deployed. The bridge conduit may be deployed, for example by inflating a balloon coupled between the bridge conduit and a catheter, or by retracting a sheath coupled to a catheter that allows expansion of a self-expanding version of the bridge conduit. When deploying the bridge conduit, x-ray imaging of radiopaque materials in the conduit framework or woven into the polymeric conduit wall may provide visual monitoring of the location and orientation of the bridge conduit. Medication on the anastomotic stent or on the conduit wall of the bridge conduit may be allowed sufficient time to begin its delivery or elution after the placement of a bridge conduit or stent in its general area of deployment, but before the actual deployment of the bridge conduit or stent.

When the bridge conduit is deployed, the bridge conduit is coupled to the side port of the first anastomotic stent at a first end of the bridge conduit and coupled to the side port of the second anastomotic stent at a second end of the bridge conduit. The bridge conduit may be coupled to the side ports with a coupling maneuver such as flaring, clasping, locking, stapling, engaging, suturing, or securing. For example, the bridge conduit may be deployed by enlarging the bridge conduit to affix the first end of the bridge conduit to a stent framework of the first anastomotic stent, and enlarging the bridge conduit to affix the second end of the bridge conduit to a stent framework of the second anastomotic stent. The length of the bridge conduit may be selected or trimmed so that the bridge conduit fits comfortably between the two anastomotic stents. Although a presently preferred embodiment utilizes catheter-delivered and connected anastomotic apparatus, portions of the procedure may be done endoscopically through, for example, the ribs to suture or otherwise attach the graft to the anastomotic stent. In another embodiment, multiple balloons on the delivery catheter are inflated simultaneously or in turn to deploy the bridge conduit.

Fluid flows through the bridge conduit, as seen at block 725. The fluid flows between the first vessel and the second vessel through one end of the first anastomotic stent, the bridge conduit, and one end of the second anastomotic stent. The fluid generally flows in one direction or the other, depending on the locations of the deployed anastomotic stents in the body.

In one exemplary embodiment of the present invention, a portion of the first or second vessel may be purposely blocked, as seen at block 730. The first or second vessel is blocked to restrict flow of the fluid through the first or second vessel, respectively. The vessels may be blocked at specific locations, for example, by clipping, stapling or suturing. A vascular plug or other suitable blocking device may be used to fully or partially block the flow of fluid so that the fluid may be routed effectively through the bridge conduit. The second vessel may be blocked, for example, upstream and downstream of two anastomotic stents to reverse the flow of fluid through the vessel when the vessel is a vein that normally carries blood flow in the opposite direction. In another example, the flow of fluid through the second vessel may be partially blocked so that some of the fluid may continue to flow through the vessel in the original direction.

In cases where a second anastomotic assembly is used, a third anastomotic stent is positioned and deployed, as seen at block 735. For example, the third anastomotic stent may be positioned opposite the occlusion in the first vessel and deployed adjacent to the occlusion. A balloon catheter or a delivery catheter with a retractable sheath may be used to deploy the third anastomotic stent. The third anastomotic stent includes a side port that is oriented in the general direction of the fourth anastomotic stent.

A fourth anastomotic stent is positioned and deployed, as seen at block 740. For example, the fourth anastomotic stent may be positioned, oriented and deployed in the second vessel using a catheter-based delivery system. The fourth anastomotic stent includes a side port, oriented generally in the direction of the side port of the third anastomotic stent.

Openings are formed in the first and second vessels, as seen at block 745. The openings are formed in the walls of the vessel adjacent to and aligned with the side ports, and may be formed within the vessel with, for example, catheter-based cutting tools, endoscopic cutting lasers, knives or scissors.

A second bridge conduit is deployed, as seen at block 750. The second bridge conduit is deployed through the second openings in the vessels by using catheter-based delivery systems. When the bridge conduit is deployed, the second bridge conduit is coupled to a side port of the third anastomotic stent at a first end of the second bridge conduit, and coupled to a side port of the fourth anastomotic stent at a second end of the bridge conduit.

Fluid flows through the second bridge conduit, as seen at block 755. In one example, the fluid flows between the first vessel and the second vessel in coordination with the first bridge conduit to bypass the occlusion. The fluid generally flows in one direction or the other through the bridge conduit, depending on the configuration of the stent assemblies in the body.

The order of deployment of the anastomotic stents, formation of openings in the vessel walls, deployment of the bridge conduits, and deployment or insertion of any vascular plugs may be altered from that described above depending on the location of the occlusions, the vessels used in the bypass, the number of anastomotic apparatus used, and the particular placement of the anastomotic stents and bridge conduits. For example, the order of stent deployment may be prescribed such that an anastomotic apparatus further away from the insertion point of the delivery catheter is deployed prior to anastomotic apparatus nearer the insertion point to avoid the need to position an anastomotic stent through a previously deployed anastomotic stent.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. An anastomotic apparatus for a bypass, comprising:
a first anastomotic stent including a side port;
a bridge conduit including a first end and a second end, the first end of the bridge conduit in communication with the side port of the first anastomotic stent; and
a second anastomotic stent including a side port, the second end of the bridge conduit in communication with the side port of the second anastomotic stent, wherein the first anastomotic stent is positionable in a first vessel and the second anastomotic stent is positionable in a second vessel, and the bridge conduit provides a pathway for fluid flow between the first vessel and the second vessel.

2. The apparatus of claim 1 wherein the anastomotic apparatus allows a fluid to flow through a first end of the first anastomotic stent, a portion of the fluid to flow through the bridge conduit, and a remaining portion of the fluid to flow through a second end of the first anastomotic stent.

3. The apparatus of claim 1 wherein the anastomotic apparatus forms an H-shape when deployed in a body.

4. The apparatus of claim 1 wherein the first anastomotic stent is positionable in an arterial vessel and the second anastomotic stent is positionable in a venous vessel.

5. The apparatus of claim 1 wherein the first anastomotic stent is positionable in a coronary artery and the second anastomotic stent is positionable in an internal thoracic artery.

6. The apparatus of claim 1 wherein the first anasotomotic stent is positionable in a femoral artery and the second anastomotic stent is positionable in one of a femoral vein or a saphenous vein.

7. The apparatus of claim 1 wherein the first anastomotic stent or the second anastomotic stent is deployable with one of a balloon catheter or a delivery sheath coupled to a catheter that retracts to deploy the anastomotic stent.

8. The apparatus of claim 1 wherein the bridge conduit is deployable with one of a balloon catheter or a delivery sheath coupled to a catheter that retracts to deploy the bridge conduit.

9. The apparatus of claim 1 wherein the first anastomotic stent or the second anastomotic stent includes a stent framework comprising a material selected from the group consisting of stainless steel, nitinol, tantalum, MP35N alloy, platinum, titanium, a chromium-based alloy, a cobalt-based alloy, a suitable biocompatible alloy, a suitable biocompatible material, a biocompatible polymer, and a combination thereof.

10. The apparatus of claim 1 wherein the first anastomotic stent or the second anastomotic stent includes a stent framework and a drug-polymer coating disposed on the stent framework.

11. The apparatus of claim 1 wherein the bridge conduit comprises a conduit framework and a polymeric conduit wall connected to at least a portion of the conduit framework.

12. A method of bypassing an occlusion in a vessel, the method comprising:
deploying a first anastomotic stent in a first vessel adjacent to an occlusion in the first vessel, the anastomotic stent including a side port;
deploying a second anastomotic stent in a second vessel, the second anastomotic stent including a side port;
forming openings in the first vessel and the second vessel, the openings aligned with the side ports;
deploying a bridge conduit through the vessel openings, wherein the bridge conduit is coupled to the side port of the first anastomotic stent at a first end of the bridge conduit and the bridge conduit is coupled to the side port of the second anastomotic stent at a second end of the bridge conduit when the bridge conduit is deployed; and
flowing fluid through the bridge conduit between the first vessel and the second vessel.

13. The method of claim 12 wherein deploying the first anastomotic stent or the second anastomotic stent comprises inflating a balloon coupled between the anastomotic stent and a catheter.

14. The method of claim 12 wherein deploying the first anastomotic stent or the second anastomotic stent comprises retracting a sheath coupled to a catheter to allow expansion of the anastomotic stent.

15. The method of claim 12 wherein deploying the bridge conduit comprises one of inflating a balloon coupled between the bridge conduit and a catheter or retracting a sheath coupled to a catheter to allow expansion of the bridge conduit.

16. The method of claim 12 wherein the bridge conduit is coupled to the side port with a coupling maneuver selected from the group consisting of flaring, clasping, locking, stapling, engaging, suturing, and securing.

17. The method of claim 12 wherein deploying the bridge conduit comprises:
enlarging the bridge conduit to affix a first end of the bridge conduit to a stent framework of the first anastomotic stent; and
enlarging the bridge conduit to affix a second end of the bridge conduit to a stent framework of the second anastomotic stent.

18. The method of claim 12 further comprising:
blocking a portion of the second vessel to restrict flow of the fluid through the second vessel.

19. The method of claim 12 further comprising:
deploying a third anastomotic stent in the first vessel adjacent to the occlusion, the third anastomotic stent opposite the first anastomotic stent;
deploying a fourth anastomotic stent in the second vessel;
forming second openings in the first and second vessels, the second openings aligned with the side ports of the third and fourth anastomotic stents;
deploying a second bridge conduit through the second openings in the vessels, the second bridge conduit coupled to a side port of the third anastomotic stent at a first end of the second bridge conduit and the second bridge conduit coupled to a side port of the fourth anastomotic stent at a second end of the second bridge conduit when the bridge conduit is deployed; and
flowing fluid through the second bridge conduit between the first vessel and the second vessel, the flow of fluid bypassing the occlusion.

20. An anastomotic stent for a bypass, comprising:
a stent framework, the stent framework having a generally tubular structure with a first end and a second end, and a side port between the first end and the second end, the side port endovascularly connectable to a bridge conduit with a coupling mechanism to provide a pathway for fluid flow through the side port and at least one end of the stent framework.

21. The anastomotic stent of claim 20 wherein the anastomotic stent is deployable with one of a balloon catheter or a delivery sheath coupled to a catheter that retracts to deploy the anastomotic stent.

22. The anastomotic stent of claim 20 further comprising:
a drug-polymer coating disposed on the stent framework.

23. The anastomotic stent of claim 20 further comprising:
a delivery catheter coupled to the anastomotic stent.
